(19) United States
(12) Patent Application Publication
(10) Pub. No.: US 2008/0177358 A1
(43) Pub. Date: Jul. 24, 2008

(54) Diagnosis and treatment methods relating to application of external heat

(75) Inventor: Scott Gammons, Loudon, TN (US)

Correspondence Address: KNOX PATENTS, P.O. BOX 30034, KNOXVILLE, TN, 37930-0034, US

(73) Assignee: ADROIT MEDICAL SYSTEMS, INC., Loudon, TN (US)

(21) Appl. No.: 12/014,859

(22) Filed: Jan. 16, 2008

Related U.S. Application Data
(60) Provisional application No. 60/885,501, filed on Jan. 18, 2007.

(51) Int. Cl. A61F 7/08

(52) U.S. Cl. 607/96

(57) ABSTRACT

A method for treating conditions responsive to heat therapy and monitoring such treatments to determine efficacy. The method includes the step of diagnosing a patient to determine if heat therapy is a proper treatment. If so, then the steps of applying heat therapy and monitoring at least one parameter associated with the patient is performed. The data collected is then trended to determine the efficacy of the heat therapy. If the efficacy indicates that the therapy is beneficial, then the steps of applying therapy, monitoring the at least one parameter, and trending the data are repeated. The heat therapy is performed by a thermal pad that applies heat with a controlled temperature to a body portion of the patient. The at least one parameter is selected from the set including perfusion index, blood oxygenation, pulse rate, local skin temperature, body core temperature, heat therapy temperature, and/or ambient temperature

### CROSS-REFERENCE TO RELATED APPLICATIONS

This application claims the benefit of U.S. Provisional Application No. 60/885,501, filed Jan. 18, 2007.

### STATEMENT REGARDING FEDERALLY SPONSORED RESEARCH OR DEVELOPMENT

Not Applicable

### BACKGROUND OF THE INVENTION

1. Field of Invention

This invention pertains to a method that includes diagnosing and treating conditions that are susceptible to benefiting from the application of heat therapy, monitoring a patient during such heat therapy, and trending the results of such heat therapy over time to determine the efficacy of the treatment.

2. Description of the Related Art

Thermal conditioning of patients is often used to control hypothermia or hyperthermia. To prevent or overcome hypothermia, heat is applied to a patient, such as by forcing warmed air through an air blanket or air mattress that has a controlled discharge of the warmed air. It is also known to force a warmed fluid, such as water, through a blanket or pad to apply heat by conduction to the patient. Inducing hyperthermia is often accomplished in a similar manner. Controlling hyperthermia is often accomplished in a similar manner except that a cooled fluid is used to draw heat away from the patient.

Certain aspects of thermal treatment of patients has been an inexact science. Home remedies have been known for a long time. For example, a dishcloth soaked in either hot or cold water is often placed against a body part to bring relief for a suffered ailment. But, a methodological treatment using heat therapy has eluded science.

### BRIEF SUMMARY OF THE INVENTION

According to various embodiments of the present invention, methods of diagnosing and/or treating conditions responsive to treatment by heat therapy and methods of monitoring such treatments are provided. Conditions that result in low blood flow or circulation and/or low blood and/or tissue oxygenation are diagnosed and then treated with heat therapy while certain variables are monitored. Such conditions include diabetes, arthritis, musculoskeletal pain management, wound therapy, cancer treatment, and post surgery infection prevention or reduction. Wound therapy applies to wounds resulting from injury, surgery, and/or pressure narcosis. The methods aid in managing pain relief and increasing blood circulation in localized areas.

In one embodiment, a thermal therapy system includes a thermal unit attached to a thermal pad that is positioned adjacent a specified area of a patient's body. A sensor monitors the effects of the thermal therapy and communicates with a processor that also communicates with the thermal unit. The thermal therapy system selectively applies a thermal treatment of a specified temperature. To determine efficacy, the system measures one or more variables. In various embodiments, the measured variables include one or more of perfusion index, blood oxygenation, pulse rate, local skin temperature, body core temperature, heat therapy temperature, and ambient temperature. The measured variables are trended and the course of treatment is determined based on the efficacy of the duration and frequency of the applied heat therapy in view of the monitored variables.

In one embodiment, the method of diagnosis and treatment includes first determining a diagnoses and the portion of the body to be treated. The method includes applying heat therapy, such as with the thermal therapy system described above. Also included in the method is measuring and monitoring one or more variables indicative of the patient's condition. The measured variables are trended to determine the efficacy of the therapy. If the therapy is beneficial, the therapy is repeated.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a schematic of one embodiment of a thermal treatment system;

FIG. 2 is a flow diagram of one embodiment of the diagnosis and treatment methodology;

FIG. 3 is a chart of test results for the right leg of a subject during one case study;

FIG. 4 is a chart of test results for the left leg of a subject during one case study;

FIG. 5 is a chart of test results for the leg of a subject with diabetes; and

FIG. 6 is a chart of test results for the arm of a subject in good health.

### DETAILED DESCRIPTION OF THE INVENTION

An apparatus for a thermal treatment system 100 and a method for the diagnosis and treatment methodology, or course of treatment, 200 is disclosed. Localized heat therapy increases blood flow in muscle tissue in skin and offers musculoskeletal pain relief. To optimize the therapy, the applied temperature must be controlled and the patient monitored to determine the efficacy of the treatment.

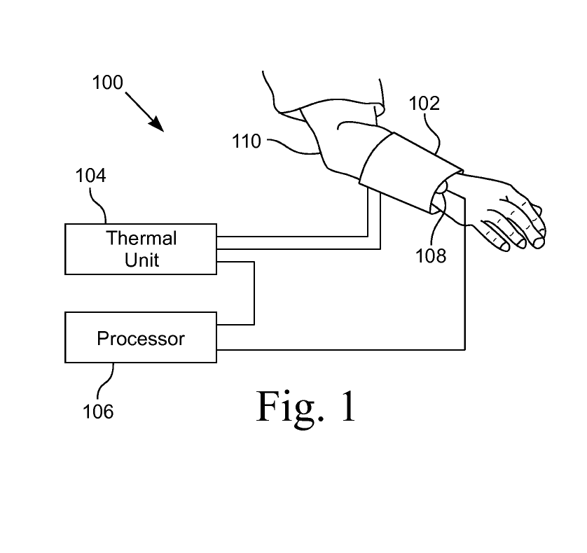

FIG. 1 illustrates a schematic of one embodiment of a thermal treatment system 100. In the illustrated embodiment, the system 100 includes a thermal unit 104 connected to a thermal pad 102. The temperature of the thermal pad 102 is determined and controlled by the thermal unit 104. The thermal unit 104 is a device that heats a fluid to a controlled temperature and pumps that fluid to the thermal pad 102. In one embodiment, the fluid is water or other liquid and the thermal unit 104 controls the temperature of the fluid within narrow limits, for example, within plus/minus 1 degree Fahrenheit.

The thermal pad 102 is positioned adjacent a body part of a patient 110. In one embodiment, the pad 102 includes a fluid conduit that follows a tortuous path over the area covered by the pad 102. The thermal unit 104 supplies a temperature controlled fluid to the pad 102, and heat transfer occurs between the patient 110 and the fluid in the pad 102. In various embodiments, the pad 102 is configured to contact or conform to a body part of the patient 110. In the illustrated embodiment, the pad 102 wraps around the forearm of the patient 110. In other embodiments the pad 102 is configured to wrap around or be placed in contact with the upper arm, a portion of the torso, a portion of a leg, or other body part desired to be treated.

Also positioned adjacent the patient 110 is at least one sensor 108. The sensor 108 is in communication with a processor, or other instrument, 106 that, in various embodiments, measures, records, compares the value with another, or otherwise processes the measured variable. In the illustrated embodiment, the processor 106 is in communication with the thermal unit 104 to control the temperature and/or the time of treatment.

The sensor 108 is a device that monitors one or more variables associated with the patient 110 and the thermal pad 102. It is understood that the sensor 108, although illustrated as a single device, in some embodiments includes multiple sensors that each measure at least one variable or parameter. In various embodiments, the sensor 108 measures the perfusion index, oxygenation, pulse rate, body core temperature, local skin temperature adjacent the pad 102, the temperature of the heat source, namely, the pad 102, and ambient temperature.

The processor 106 receives inputs from the sensor 108. In one embodiment the processor 106 is a general purpose computer, in other embodiments, it is a specialized device, either analog or digital, for implementing the functions of the invention. Those skilled in the art will recognize that the processor 106 includes an input component, an output component, a storage component, and a processing component. The input component receives input from external devices, such as the sensor 108. The output component sends output to external devices, such as the thermal unit 104. The storage component stores data and program code. In various embodiments, the storage component includes random access memory, non-volatile memory, such as floppy disks, hard disks, and writeable optical disks. The processing component executes the instructions included in the software and routines. In one embodiment, the processor 106 stores the data representing the measured parameters from the sensor 108. In one embodiment, the processor 106 executes software that trends the data from multiple therapy sessions. The data from individual therapy sessions is stored by the processor 106 and the data is used for trend analysis to determine the efficacy of the treatment. In another embodiment, the processor 106 is one or more computers that store and process the data from the measured parameters.

In the illustrated embodiment, the processor 106 is in communication with the thermal unit 104. In various embodiments, the processor 106 and thermal unit 104 share data to control the thermal unit 104 within limits based on the measured parameters from the sensor 108. For example, when the sensor 108 is measuring the temperature of the pad 102, the processor 106 provides information to the thermal unit 104 to maintain the measured temperature within limits. In another example, when the sensor 108 is measuring the temperature of the patient 110 adjacent the pad 102, the processor 106 provides information to the thermal unit 104 to maintain the temperature of the pad 102 so that the thermal treatment maintains the patient temperature within limits.

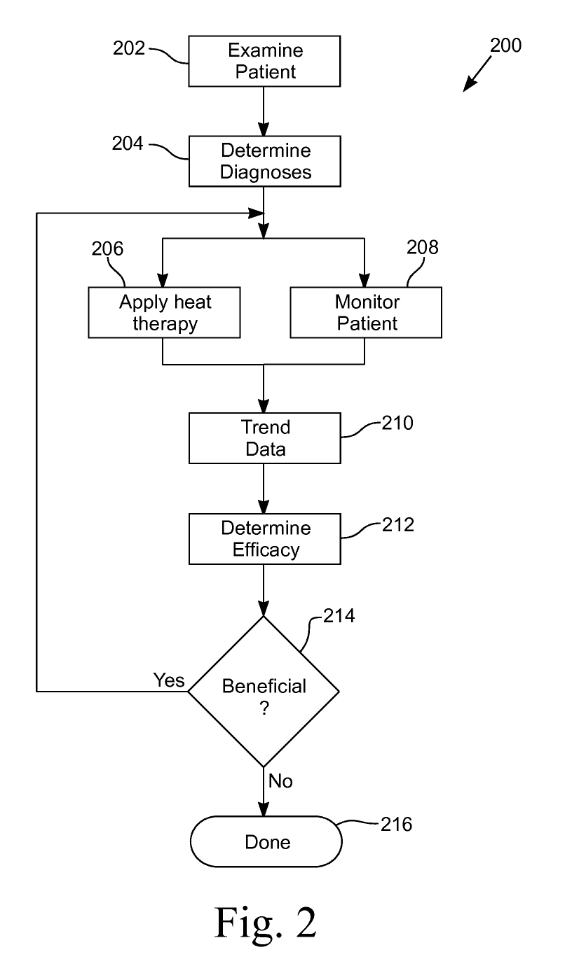

FIG. 2 illustrates a flow diagram of one embodiment of the diagnosis and treatment methodology, or course of treatment, 200. The first step 202 is to examine a patient 110. The next step 204 is to determine the diagnoses of the disease or ailment based on the examination of the patient 110. It is noted that the patient 110 is not limited to humans, but more generally includes animals. In one embodiment, the steps 202, 204 of examining and diagnosing the patient 110 includes determining the desired temperature and body portion for application of the heat therapy.

In one embodiment, the condition to be treated includes diabetes. The steps 202, 204 of examining and diagnosing the patient 110 include the standard accepted tests for diagnosing diabetes. The steps 202, 204 also include identifying the portions of the patient's body that are at most risk of damage and/or the portions of the patient's body that would be most receptive to heat therapy treatment. For example, diabetics often suffer from poor blood circulation, particularly at the extremities, including the limbs, fingers, and toes. It has been discovered that heat therapy increases the blood flow and oxygenation of the tissues subcutaneously adjacent the treatment areas, which is beneficial to the health of those suffering from diabetes.

In another embodiment, the condition to be treated is arthritis. The steps 202, 204 of examining and diagnosing the patient 110 include the standard accepted tests for diagnosing arthritis. The steps 202, 204 also include identifying the portions of the patient's body that would be most receptive to heat therapy treatment. It has been discovered that heat therapy increases the blood flow and the oxygenation of the tissues subcutaneously adjacent the treatment areas, which is beneficial to relieving pain suffered by suffering from arthritis.

In still another embodiment, the condition to be treated is musculoskeletal pain management. The steps 202, 204 of examining and diagnosing the patient 110 include identifying the portion of the patient's body where heat therapy is to be applied such that the application of heat reduces or eliminates the pain and/or discomfort experienced by a patient.

In still another embodiment, the condition to be treated is a wound, such as resulting from injury, surgery, and/or pressure narcosis. The steps 202, 204 of examining and diagnosing the patient 110 include identifying the portion of the patient's body where heat therapy is to be applied. In one embodiment, the body portion to be treated includes the location of the wound and a portion of the surrounding skin. In another embodiment, the body portion to be treated includes the skin surrounding the wound, but does not include the wound itself.

In still another embodiment, the condition to be treated is cancer. The steps 202, 204 of examining and diagnosing the patient 110 include identifying the portion of the patient's body where heat therapy is to be applied. In one embodiment, the body portion to be treated includes the skin surface adjacent to and surrounding a cancerous growth. In another embodiment, the body portion to be treated includes a substantial portion of the body, which results in the core temperature of the patient increasing above normal body temperature. It has been found that heat therapy that elevates the body temperature and simulates feverish conditions serves to stimulate the auto-immune system of the patient 110. Additionally, it has been found that heat therapy directed to cancerous growths, if the heat itself does not kill the cancerous cells, the heat effectively increases the cancerous cells' sensitivity to treatment by other modalities.

In still another embodiment, the condition to be treated is post-surgical infection prevention or reduction. The steps 202, 204 of examining and diagnosing the patient 110 include identifying the portion of the patient's body where heat therapy is to be applied to increase the amount of oxygenation of the tissues surrounding the incision created during surgery. It has been discovered that heat therapy increases the blood flow and the oxygenation of the tissues subcutaneously adjacent the treatment areas, which is beneficial by increasing the number of antibodies adjacent the incision that are available for warding off post-surgical infection.

After the steps 202, 204 of examining and diagnosing the patient, the steps of applying heat therapy 206 and monitoring 208 the patient 110 are performed. In various embodiments, the steps 206, 208 of applying heat therapy and monitoring are performed simultaneously, one before the other, or the step 208 of monitoring is performed before and after the step 206 of applying heat.

The step 206 of applying heat therapy, in one embodiment, includes applying or wrapping a pad 102 around a body portion of a patient 110. A heated fluid is pumped through a tortuous conduit inside the pliable pad 102. The fluid temperature is controlled within a narrow range. A fluid-based pad 102 applied to the body portion of the patient has the advantage of providing a relatively constant temperature across a large surface area of the patient because of the high specific heat and thermal conductivity of the water or other liquid used in the liquid fluid-based pad 102. Also, the use of a liquid fluid allows for more precise control of the temperature. In one embodiment, 30 to 60 minutes of low level heat therapy treatment between 100 and 107 degrees Fahrenheit repeatedly produces a five-fold increase in perfusion index levels. In other embodiments, effective fluid temperatures include values less than 104 degrees Fahrenheit, which is less than the accepted clinical rule of thumb for heat therapy temperature. In another embodiment, the heat therapy temperature is at a temperature at or above 75 degrees Fahrenheit. In such an embodiment, it is noted that the normal body temperature at the skin is approximately 90 degrees Fahrenheit, but that a person with poor circulation has a skin temperature that is less than the normal body temperature. Accordingly, heat therapy is applied at a temperature that is above the temperature of the patient's skin in the area in which the heat therapy is to be applied.

The step 208 of monitoring the patient 110, in various embodiments, includes measuring one or more of the perfusion index, the blood oxygenation, pulse rate, local skin temperature, body core temperature, heat therapy temperature, and ambient temperature. The step 208 of monitoring the patient 110 allows the step 206 of applying the heat therapy to continue as long as it is beneficial to the patient and at the lowest temperature that produces results. For example, when the diagnosis is to prevent or reduce the post-surgical infection rate, the oxygenation adjacent the incision area is monitored to ensure it is at an adequate level to avoid infection. The step 208 of monitoring the patient 110 allows the temperature of the heat therapy to be as low as possible to ensure the comfort of the patient for the extended time that the heat therapy is applied. In various embodiments, heat therapy is applied 206 in such cases for periods extending from several hours to several days.

Through the step 208 of monitoring the patient 110, the step 206 of applying heat therapy is controlled to produce optimum results and provide for the comfort of the patient 110. The temperature and the duration of the heat therapy are controllable. By maintaining the temperature of the heat therapy at the lowest possible temperature that still produces results, the patient 110 is not exposed to the discomfort caused by a hot pad 102. Also, by controlling the duration of the step 206 of applying heat therapy, the length of time that the patient 110 is subject to any discomfort caused by the heat therapy is minimized to that time in which the heat therapy provides the necessary results as indicated by the monitored variables.

After the steps 206, 208 of applying heat therapy and monitoring the patient 110, the steps 210, 212 of trending the data and determining the treatment efficacy are performed. In one embodiment, the steps 210, 212 of trending the data and determining the treatment efficacy are performed with the aid of a processor 106. After the steps 210, 212 of trending the data and determining the treatment efficacy, the step 214 of determining if the treatment is beneficial is performed. If the course of treatment 200 is beneficial, the steps 206, 208 of applying heat therapy and monitoring the patient 110 are repeated. If the data indicates that the course of treatment 200 is no longer beneficial, the treatment 200 is done, or complete, 216.

The step 210 of trending the data allows the progress of the treatment 200 to be stored for evaluation during the step 212 of determining the treatment efficacy. The step 210 of trending also includes comparing the current data from the step 208 of monitoring the patient 110 to be compared for changes from the historical data from previous occurrences of the steps 206, 208 of applying heat therapy and monitoring the patient 110.

The step 212 of determining the treatment efficacy includes determining from the step 210 of trending if the changes over time of the measured parameters from the step 208 of monitoring the patient 110 show an improvement in the condition of the patient 110. The set of data trended includes the measured variables from step 208 of monitoring the patient 110 for different therapy sessions. That is, the first time that steps 206, 208 of applying heat and monitoring are performed the data is stored as the set of data. The second and subsequent times the steps 206, 208 of applying heat and monitoring are performed, such as for different therapy sessions, the new data is added to the set of data and the set of data includes the new data and historical data.

In one embodiment, the set of data to be trended in step 210 includes the initial data on the patient 110 obtained during the step 202 of examining the patient. This initial data is the historical data that is used for step 210 of trending where the newly acquired data from step 208 is compared to the initial data. The step 212 of determining efficacy includes comparing the newly acquired data to the initial data to determine if there has been a change in any measured parameter.

The step 214 of determining if the heat therapy has beneficial effects includes determining if the change in any measured parameter indicates an improvement in the patient's condition. Efficacy is shown and the step 206 of applying heat therapy has beneficial effects when the perfusion index increases. The step 206 of applying heat therapy has beneficial effects on the oxygenation of tissue when that measured variable increases over time. If the time between the step 206 of heat therapy is such that the general trend of the starting value of the measured variables are increasing for each occurrence of the step 206 of heat therapy, then the efficacy of the treatment is shown and the step 214 of determining if the treatment is beneficial has a positive result.

The steps 210, 212, 214 of trending the data and determining the treatment efficacy and then determining if the treatment is beneficial allow the treatment 200 to proceed in a manner that results in a general increase in the health of the patient 110. For example, the perfusion index increases during the step 206 of applying heat therapy and continues at an elevated level for a period of time after the step 206 is completed. The steps 208, 210 of monitoring and trending allow the determination of the optimum duration and frequency of the step 206 of applying heat therapy in order to provide optimum treatment of the patient 110. In particular, the step 212 of determining the treatment efficacy allows the customization of the treatment 200 to accommodate the duration and frequency of the step 206 of applying heat therapy.

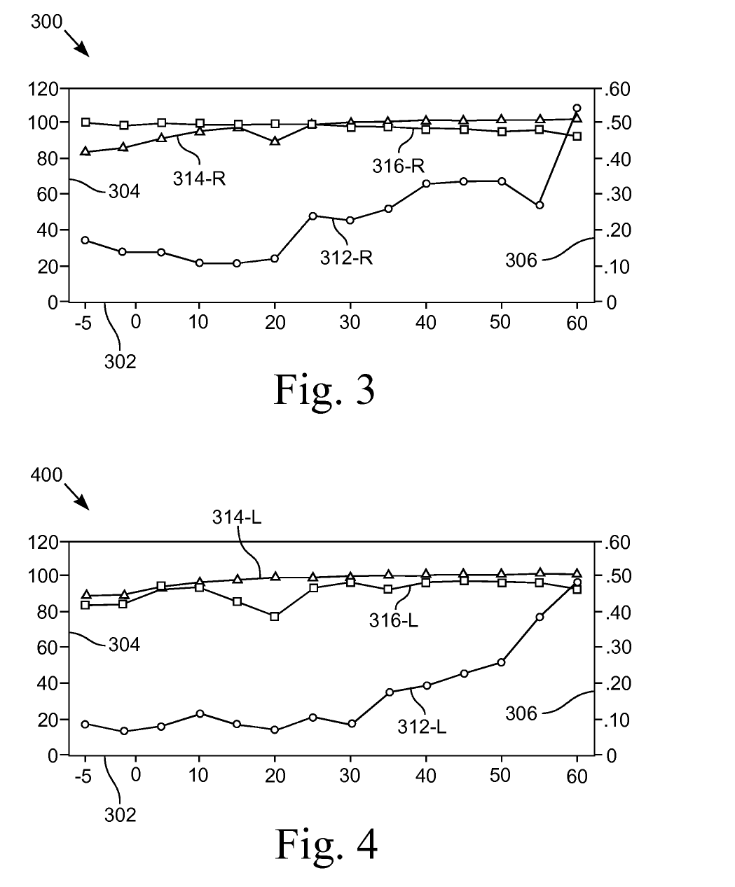

FIG. 3 charts the test results for the right leg of a subject during one case study. FIG. 4 charts the test results for the left leg of a subject during one case study. The results for each leg were obtained on different days. The charts 300, 400 include an x-axis 302 of time with a left y-axis 304 showing the units of oxygenation 316 and skin temperature 314 and a right y-axis 306 showing the units for perfusion index 312.

In the case study, a 58 year old female subject with a right leg that was slightly withered and a left leg that was healthy was monitored during treatment of each limb. The subject's right leg, which had undergone eight surgeries in the previous seventeen years, had skin discoloration from subcutaneous collection of blood. The subject was not known to have diabetes. Each leg was individually treated with heat therapy while being monitored. The variables being monitored for each leg included skin temperature 314-R, 314-L, blood oxygenation (SpO2) 316-R, 316-L, and perfusion index 312-R, 312-L.

The test methodology was to monitor the variables 312, 314, 316 starting five minutes before the heat therapy began. At that time, the therapy pad was wrapped around the lower portion of the subject's leg being studied. At time zero, heat therapy began with the application of heat to the wrapped leg. Heat therapy continued for 60 minutes with the pad temperature maintained at approximately 106.5 degrees Fahrenheit during the test.

Table 1 shows the raw data collected during the test of the right leg.

TABLE 1

| | | Pulse | Perf. | Pad | Skin | Room |
|---|---|---|---|---|---|---|
| Minutes | SpO2 | Rate | Index | Temp. | Temp. | Temp. |
| −5 | 100 | 97 | 0.17 | — | 83.6 | 74 |
| 0 | 98 | 93 | 0.14 | — | 86.4 | 74 |
| 5 | 100 | 96 | 0.14 | 106.3 | 91.7 | 74 |
| 10 | 99 | 95 | 0.11 | 106.5 | 95.7 | 74 |
| 15 | 99 | 96 | 0.11 | 106.5 | 97.7 | 74 |
| 20 | 100 | 100 | 0.12 | 106.5 | 89.9 | 74 |
| 25 | 99 | 99 | 0.24 | 106.5 | 100.0 | 75 |
| 30 | 98 | 95 | 0.23 | 106.5 | 100.7 | 75 |
| 35 | 98 | 92 | 0.26 | 106.5 | 101.3 | 75 |
| 40 | 97 | 94 | 0.33 | 106.5 | 101.6 | 75 |
| 45 | 97 | 98 | 0.34 | 106.7 | 101.8 | 75 |
| 50 | 95 | 92 | 0.34 | 106.5 | 102.2 | 75 |
| 55 | 97 | 99 | 0.27 | 106.5 | 102.3 | 75 |
| 60 | 93 | 90 | 0.54 | 106.3 | 102.5 | 75 |

Table 2 shows the raw data collected during the test of the left leg.

TABLE 2

| | | Pulse | Perf. | Pad | Skin | Room |
|---|---|---|---|---|---|---|
| Minutes | SpO2 | Rate | Index | Temp. | Temp. | Temp. |
| −5 | 84 | 100 | 0.09 | — | 89.6 | 75 |
| 0 | 85 | 95 | 0.07 | — | 90.1 | 75 |
| 5 | 95 | 98 | 0.08 | 102.5 | 94.1 | 75 |
| 10 | 94 | 103 | 0.12 | 102.9 | 96.6 | 75 |
| 15 | 86 | 98 | 0.09 | 103.1 | 98.0 | 75 |
| 20 | 78 | 105 | 0.07 | 105.0 | 99.1 | 75 |
| 25 | 94 | 102 | 0.11 | 104.1 | 99.8 | 75 |
| 30 | 97 | 104 | 0.09 | 104.7 | 100.4 | 75 |
| 35 | 93 | 100 | 0.18 | 104.9 | 100.7 | 75 |
| 40 | 97 | 100 | 0.20 | 104.7 | 100.9 | 75 |
| 45 | 98 | 101 | 0.23 | 105.0 | 100.9 | 75 |
| 50 | 97 | 99 | 0.26 | 105.0 | 100.9 | 75 |
| 55 | 96 | 103 | 0.39 | 105.5 | 101.1 | 75 |
| 60 | 94 | 100 | 0.49 | 105.2 | 101.1 | 75 |

The monitored data is charted in FIGS. 2 and 3. Similar data was collected for each leg. The perfusion index 312 for each leg increased virtually the same amount despite the fact each leg had a different beginning and ending value. Higher perfusion index values 312-R in the right leg are believed caused by the visible subcutaneous collection of blood over which the sensor was placed. As such, the prevailing condition skewed right leg perfusion index values 312-R making them appear stronger than left leg perfusion index values 312-L. The subject's right leg was clearly not as healthy as the left leg. Therefore, the perfusion index values 312-R in the right leg are interpreted as relative and not an absolute indicator of health. Still, improvement in perfusion index values 312 was consistent for both legs. Interestingly the oxygenation saturation (SpO2) 316-R was also higher in the right leg and did not increase with the application of heat therapy. SpO2 316-L in the left leg rose steadily in the leg once heat therapy began. It is noted that the pulse rate was lower during right leg testing. No observations were made as to potential reasons for this, other than that the tests were conducted on different days.

Infrared images were made before and after each test on each leg. The infrared images show strikingly different skin surface temperatures of the legs. Before applying heat therapy, the subject's skin temperature for the tested leg was approximately 81° F., which is 10° F. less than the average skin temperature for a healthy person. This was also true for the right leg, which was discolored from subcutaneous collection of blood.

These test results demonstrate that the heat therapy amply increased the amount of arterial blood flow to each tested leg, regardless of the general health of the leg. These test results also demonstrate that consistent measurements of oxygenation 316 and perfusion index 312 using skin contact instruments were obtainable despite the obtrusion of a subcutaneous collection of blood.

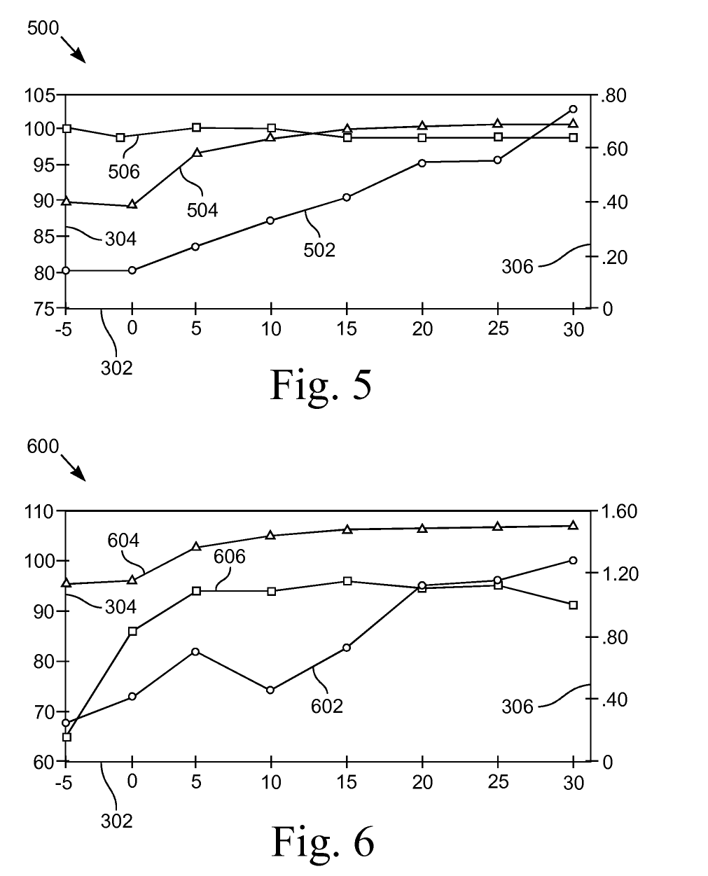

FIG. 5 illustrates a case study chart 500 of test results for the leg of a subject with diabetes. The chart 500 includes an x-axis 302 of time with a left y-axis 304 showing the units of oxygenation 506 and skin temperature 504 and a right y-axis 306 showing the units for perfusion index 502.

In the case study, the subject was a 63 year old female who had been diagnosed two year earlier with Type II Diabetes. The subject's legs had been burnt several years before then.

Table 3 shows the raw data collected during the test.

TABLE 3

| | | Pulse | Perf. | Pad | Skin | Room |
|---|---|---|---|---|---|---|
| Minutes | SpO2 | Rate | Index | Temp. | Temp. | Temp. |
| −5 | 100 | 92 | 0.15 | — | 89.9 | 70 |
| 0 | 99 | 96 | 0.15 | — | 89.7 | 70 |
| 5 | 100 | 94 | 0.24 | 102.3 | 96.8 | 72 |
| 10 | 100 | 94 | 0.34 | 103.2 | 99.3 | 72 |
| 15 | 99 | 94 | 0.42 | 103.6 | 100.4 | 72 |
| 20 | 99 | 98 | 0.55 | 103.8 | 100.9 | 72 |
| 25 | 99 | 99 | 0.56 | 104.0 | 101.3 | 72 |
| 30 | 99 | 96 | 0.75 | 104.0 | 101.3 | 72 |

The volunteer's oxygen saturation (SpO2) 506 reading prior to therapy was 100 and did not increase during the test. However, the perfusion index (PI) 502 was low prior to therapy, but showed a steady and substantial increase following the application of heat. The initial perfusion index value 502 was 0.15 and climbed 500% to 0.75 after 30 minutes of heat therapy. Perfusion index 502 is a relative measurement of pulse strength and an indication of the amount of blood delivered to the skin and tissue. This test indicates that the heat therapy treatment amply increased the amount of arterial blood flow to the diabetic limb.

FIG. 6 illustrates a chart 600 of test results for the arm of a subject in good health. The chart 600 includes an x-axis 302 of time with a left y-axis 304 showing the units of oxygenation 606 and skin temperature 604 and a right y-axis 306 showing the units for perfusion index 602. In the case study, the subject was a 53 year old female who was in good heath and not known to be diabetic. The subject was tired and sleepy throughout the test.

Table 4 shows the raw data collected during the test.

TABLE 4

| | | Pulse | Perfusion | Pad | Skin | Room |
|---|---|---|---|---|---|---|
| Time | SpO2 | Rate | Index | Temp. (F.) | Temp. | Temp. |
| 2:57:21 | 64 | 75 | 0.27 | — | 95.7 | 78 |
| 3:01:05 | 86 | 75 | 0.44 | — | 96.4 | 79 |
| 3:06:01 | 93 | 78 | 0.69 | 106.7 | 101.4 | 79 |
| 3:11:01 | 93 | 78 | 0.45 | 106.3 | 102.5 | 79 |
| 3:16:05 | 96 | 78 | 0.71 | 106.3 | 103.1 | 79 |
| 3:21:01 | 94 | 79 | 1.12 | 106.5 | 103.2 | 79 |
| 3:26:01 | 95 | 79 | 1.18 | 106.5 | 103.4 | 80 |
| 3:31:01 | 91 | 80 | 1.28 | 106.7 | 103.6 | 80 |

The volunteer's oxygen saturation (SpO2) 606 and perfusion index (PI) 602 increased steadily and significantly after the initiation of localized heat therapy. The increase in SpO2 606 was an average of 8.2% with a peak of 10.5%. The perfusion index 602 (a relative measurement of pulse strength) also increased an average of 205% with a peak increase of 290% at 30 minutes of heat therapy. This test indicates that there is a measurable correlation between controlled localized heat therapy and increased oxygen content.

Other tests have shown an average increase of the perfusion index. One such test showed a perfusion index increase of 117% after 30 minutes of treatment and 148% after 60 minutes of treatment. The residual effect after 30 minutes is an average of 31%. Another such test showed an increase of the perfusion index of 63% after 30 minutes of treatment with a residual effect of 9% after 30 minutes.

From the foregoing description, it will be recognized by those skilled in the art that a method of diagnoses and a method of treatment using heat therapy has been provided. The method includes determining a body portion to be treated, applying a heat source to that body portion, monitoring and measuring at least one parameter associated with the heat source, trending the data accumulated from the measured parameters, and determining the efficacy of the treatment to date.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. A method of treatment using a heat therapy device, said method comprising the steps of:
a) determining a body portion of a patient to receive heat therapy;
b) applying a thermal pad to said body portion of the patient for a specified time, said thermal pad providing heat at a controlled temperature;
c) monitoring at least one parameter associated with said step b) of applying said thermal pad;
d) after said steps of b) applying and c) monitoring, trending a set of data associated with said at least one parameter;
e) determining an efficacy for said step b) of applying from said step d) of trending said set of data; and
f) if said efficacy indicates said step b) of applying a thermal pad was beneficial, repeating said steps b) to e).

2. The method of claim 1 wherein said step c) of monitoring includes storing said at least one parameter with said set of data.

3. The method of claim 1 wherein said step c) of monitoring includes measuring a perfusion index of the patient.

4. The method of claim 1 wherein said step c) of monitoring includes measuring said at least one parameter selected from the group including a perfusion index, an oxygenation level, a pulse rate, a body core temperature, a local skin temperature adjacent said body portion, a temperature of said thermal pad adjacent to said body portion, and an ambient temperature.

5. The method of claim 1 wherein said set of data includes historical data from at least one previous occurrence of step b) of applying a thermal pad, and said step d) of trending includes determining a change of said at least one parameter relative to said historical data.

6. The method of claim 1 further including a step of examining the patient before said step b) of applying said thermal pad, said step of examining including measuring said at least one parameter and saving said at least one parameter as historical data in said set of data, and wherein said step d) of trending includes determining a change of said at least one parameter relative to said historical data.

7. The method of claim 1 wherein said efficacy determined in said step e) is beneficial if said at least one parameter indicates an improvement over time.

8. A method of treatment using a heat therapy device, said method comprising the steps of:
a) determining a body portion of a patient to receive heat therapy;
b) applying heat to said portion of the body of the patient;
c) monitoring at least one parameter associated with said step b) of applying heat;
d) after said step of b) applying heat and step c) of monitoring, determining an efficacy for said step b) of applying heat from a set of data associated with said at least one parameter; and
e) if said efficacy indicates said step b) of applying heat was beneficial, repeating said steps b) to e).

9. The method of claim 8 wherein said step b) of applying heat includes applying a thermal pad to the patient, said thermal pad providing heat at a controlled temperature.

10. The method of claim 8 wherein said step c) of monitoring includes measuring a perfusion index of the patient.

11. The method of claim 8 wherein said step c) of monitoring includes measuring said at least one parameter selected from the group including a perfusion index, an oxygenation level, a pulse rate, a body core temperature, a local skin temperature adjacent said body portion, a temperature corresponding to a heat source applied to said body portion, and an ambient temperature.

12. The method of claim 8 wherein said step c) of monitoring includes storing said at least one parameter with said set of data.

13. The method of claim 8 wherein said step d) of determining said efficacy includes the step of trending said set of data associated with said at least one parameter.

14. The method of claim 8 wherein said step d) of determining said efficacy includes the step of trending said set of data associated with said at least one parameter, said efficacy being beneficial if said at least one parameter indicates an improvement over time.

15. The method of claim 8 wherein said set of data includes historical data from at least one previous occurrence of step b) of applying heat, and said step d) of determining said efficacy includes determining a change of said at least one parameter relative to said historical data.

16. The method of claim 8 further including a step of examining the patient before said step b) of applying heat, said step of examining including measuring said at least one parameter and saving said at least one parameter as historical data in said set of data, and wherein said step d) of determining said efficacy includes determining a change of said at least one parameter relative to said historical data.

17. The method of claim 8 wherein said efficacy determined in said step d) is beneficial if said at least one parameter indicates an improvement over time.
